(19) United States
(12) Patent Application Publication
(10) Pub. No.: US 2010/0204326 A1
(43) Pub. Date: Aug. 12, 2010

(54) NOVEL AGENTS FOR TREATMENT OF AILMENTS AND DYSFUNCTIONS

(75) Inventor: Cletus J. M. D'Souza, Mangalore (IN)

Correspondence Address: COOK ALEX LTD, SUITE 2850, 200 WEST ADAMS STREET, CHICAGO, IL, 60606, US

(73) Assignee: Regain Biotechnology PVT LTD, Mumbai (IN)

(21) Appl. No.: 12/678,189

(22) PCT Filed: Sep. 8, 2008
(86) PCT No.: PCT/IN08/00572

§ 371 Date: Mar. 15, 2010

(30) Foreign Application Priority Data
Sep. 6, 2007 (IN) 1703/MUM/2007

(51) Int. Cl. A61K 31/20; C07C 59/185; A61P 31/10; A61P 35/04; A61P 17/00

(52) U.S. Cl. 514/558; 554/121

(57) ABSTRACT

The present invention is related to the use of novel agents effective for differential killing of abnormal cells such as cancer cells without damaging or being toxic to normal cells. Further these agents may be used for treating a host of ailments including various types of cancers, skin diseases, prevention and reversal of ageing process, prevention of inflammatory reactions, cure of bacterial infections, cure of fungal infections, etc. The agents are prepared by air oxidation of natural oils optionally in the presence of a catalyst wherein the isolated agents have Iodine value 40-60% of the starting oil, Saponification value 20-60 higher than that of the starting oil, Peroxide value 2-3 times that of the starting oil. These agents on saponification and acidification yield free fatty acids and ether linked fatty acid dimmer wherein the dimmer also acts as an active.

### FIELD OF THE INVENTION

The present invention is related to the use of novel agents effective for differential killing of abnormal cells such as cancer cells without damaging or being toxic to normal cells. Further these agents may be used for treating a host of ailments including various types of cancers, skin diseases, prevention and reversal of ageing process, prevention of inflammatory reactions, cure of bacterial infections, cure of fungal infections, etc.

### BACKGROUND OF THE INVENTION

A variety of approaches have been made to control and cure cancer involving proto oncogenes the antioncogenes and the suicide genes (Portsmouth D. Hlavaty J. and Renner M. 2007, Mol. Aspects. Med 28:4-41) The general approach has been to detect cancer at an early stage, and destroy the cancer cells. The second has been to interfere in the replication of DNA, the third has been to prevent supply of blood to the newly formed tumours and the recent approaches have been to strengthen the immune system to fight against the transformed cells.

In the case of Antiproliferative Therapy, drugs that act on rapidly dividing cells are most effective during the S phase of cell cycle generally causing DNA damage that may initiate apoptotic cell death but not without associated side effects thereby becoming toxic to bone marrow cells, impairing wound healing, damaging hair follicles and gastro-intestinal epithelium or the drugs themselves being carcinogenic [Hans-Peter Lipp, 1999.].

Conventional therapy for cancer has not been highly successful for a variety of reasons. Generally, cancer chemotherapy is painful and debilitating. Often it is ineffective, or its effect on prolonging survival is only short. Further they are expensive. Despite major advances in the basic understanding of carcinogenesis, metastasis, and angiogenesis, most of the discoveries in these fields have not been applicable in regular therapeutic practice.

In an article in J. Cell Biochem 58, 175 (1995) King and Cidlowski have used acridone derivatives to interfere in different phases of the cell cycle, which has resulted in the induction of apoptosis. However such studies have met with limited success as factors that could induce the apoptotic pathways in the cancer cells could also turn them on in normal cells resulting in low differential killing thereby leading to extensive tissue degeneration in the cancer patients. Also, when tested in vivo, it was found that the hormones of the animal could rescue the cancer cells from drug induced apoptotic killing (Thimmaiah, P and DSouza C, 2003 PhD thesis)

Skin diseases account for about 13% of all disorders and a quarter of all occupational diseases. Dermatitis is the second most common cause of occupational disease. Conditions such as contact dermatitis, folliculitis, acne pigmentary disorders and neoplasms are generally treated by topical applications of therapeutic molecules such as salicylic acid, zinc oxide, variety of antibiotics with antibacterial or antifungal functionalities, steroids generally in appropriate delivery systems [Ostrenga, J. Steinmetz, C. and Poulsen, B. J. Pharmaceutical Sci. 60:1175-1179, 2006]. However it is well established that chronic skin diseases generally do not respond to conventional treatments. While antibiotics are capable of controlling bacterial or fungal infections, development of drug resistance in the long term create limitations for the treatment of chronic skin diseases. Also clinicians are concerned about the unknown risks of using many of the topical medications (McNeill A. M. and Koo J Y. Int. J. Dermatol. 46: 656-658, 2007)

Ageing is a complex physiological process involving a variety of metabolic reactions. There are no specific diagnostic markers of ageing or prevention of aging at a molecular level, although collagen synthesis used as a marker and antioxidants have been applied for the prevention of aging process. The results from such attempts continue to be inconclusive.

In the case of treatment of inflammatory processes, steroidal anti-inflammatory molecules and nonsteroidal anti-inflammatory molecules have been used. While steroids prevent inflammatory pathways, because of the side effects it is not a preferred therapeutic molecule. Among the non-steroidal anti-inflammatory molecules aspirin is predominant. The aspirin and cyclooxygenease inhibitors act downstream to the production of arachidonic acid. However, to date no efficient anti-inflammatory molecule, has been found that acts at the level of inhibiting cellular pholipase A₂. Efficient PLA₂ inhibitors are not yet found. Efficient PLA₂ inhibitors would act as an efficient anti-inflammatory molecule. (Rainsford K. D. Subcell. Biochem. 42:3-27, 2007)

It has been a long standing need to provide effective agents to tackle diverse ailments including various types of cancers, skin diseases, prevention and reversal of ageing process, prevention of inflammatory reactions, cure of bacterial infections, cure of fungal infections, etc.

### SUMMARY OF THE INVENTION

The main object of the invention is to provide effective agents for differential killing of abnormal cells such as cancer cells without damaging or being toxic to normal cells so that they can be used for treating several ailments including various types of cancers, skin diseases, prevention and reversal of ageing process, prevention of inflammatory reactions, cure of bacterial infections, cure of fungal infections, etc.

Another object of the inventions is to provide effective agents for differential killing of abnormal cells without being significantly toxic to normal cells even at 1000 times the concentration that was required to kill the cancer cells.

Another object of the invention is to provide effective antidermatitis agents that would partition into the membranes of the skin cells in a facile manner so that they can be used to treat chronic skin diseases.

Yet another object of the invention is to provide agents that reduce the thickness of the epidermis, which is a desirable trait in maintenance of skin quality.

Yet another object of the invention is to provide agents to function as controlled PLA₂ inhibitors recognizing the fact that basal generation of eicosanodis is essential for normal cellular function.

It is yet another object of the invention to provide agents with anti bacterial and antifungal activities within minimal risk of the bacteria or fungi developing resistance to the said agents.

Yet another object of the invention is to explore natural sources for such agents for the applications mentioned above.

Yet another object of the invention is to develop processes for the extraction of the effective agents from diverse natural sources.

Thus in accordance with this invention, extracts are prepared from promising natural sources, characterized and then tested for diverse functionalities as potential candidates for the treatment of various diseases and/or abnormal physiological conditions.

### DETAILED DESCRIPTION OF THE INVENTION

### 1. Preparation of the Agents

Various natural sources such as sunflower oil, safflower oil, ground nut oil, soybean oil, sesame oil and other unsaturated oils may be selected for the preparation of the agents of this invention.

In a specific embodiment of this invention Sesame oil was selected as the preferred natural source for the preparation of the novel agents. Sesame oil was aged in a vessel optionally layered with fine layer of carbon particles, optionally in the presence of activated clay. The sesame oil undergoes auto oxidation during the process of ageing to transform to a red viscous liquid which when further aged forms a solid white material. The oxidative process may be catalysed by addition of metal oxides such as ferric oxide, lead oxide, mercury oxide, wherein lead oxide is the preferred choice. The ageing process may also be done by bubbling oxygen or air in the vessel. Further the process may be accelerated by the addition of the red viscous oil and or the said white material formed in the sesame oil during the process of ageing.

The red viscous liquid and the white material obtained during the ageing process function as agents with distinctive effects on cell cultures and for the diverse treatments mentioned above.

The red viscous liquid and the white material may further be subjected to saponification followed by acidification to obtain another set of active agents. These active agents may also be further derivatized to form phospholipids, which may then be used to form liposomes as effective delivery agents.

In addition to the saponification product the red viscous oil and the white material contain Glycerol, and fatty acids in ratios of 16:0, 18:0, 18:1, and 18:2 measured by gas chromatography. The other fatty acids with higher saturation and longer chains may be present in traces.

The saponified product can be converted to stearic acid by treatment with dry hydriodic acid followed by zinc-acetic acid reduction. The saponified product had a characteristic fluorescence excitation at 375 nm and emission at 440 nm. Some of the characteristics of the prepared agents by the above process are given in tables 1-3.

### EXAMPLES

### Preparation of Products from Sesame Oil and their Characterisation

50 ml of potassium octoate was taken in a freshly carbon coated container to which 500 ml of sesame oil was added with stirring. The container was loosely covered and ariel oxidation was allowed to take place, with or without bubbling of air till a red viscous oil with Iodine value 32, Saponification value 260 and Peroxide value 18 is separated settled at the bottom. Further ariel oxidation results in a white precipitate settled at the bottom of the container which was separated.

In one of the embodiments, the oxidation of the sesame oil was carried out in the presence of Pb₃ O₄ or calcium octoate instead of potassium octoate. In yet another embodiment, the white precipitate is further oxidized in sesame oil in the presence of Pb₃ O₄ to produce the red viscous liquid.

### Characterisation of the Products

The red viscous oil and the white product is identified as ether linked fatty acid dimer derivative.

### Saphonification of the Agents:

The red viscous oil or the white product is saponified. The saponification reaction mixture is acidified. The unreacted oil would yield free fatty acid and the red viscous oil and the white product would yield free fatty acids and the ether linked fatty acid dimer. The free fatty acids and the ether linked fatty acid dimer can be separated in many different ways. The mixture can be dissolved in ethanol or chloroform and separated on a column of Sephadex LH 20 or Sephadex LH 50. Two peaks are obtained, the first peak corresponds to the ether linked dimer and the second peak to the free fatty acids.

As another option, the fatty acids and the ether linked dimer are methylated using diazomethane. The methyl esters are distilled. The lower boiling fraction is the fatty acid methyl esters and the higher boiling fraction is the methyl ester of ether linked fatty acid dimer. The methyl esters are removed by saponification followed by acidification and extraction into solvent ether.

In yet another option, the fatty acids and the ether linked fatty acid dimer can be distilled directly under reduced pressure by applying vacuum. Fatty acid fraction boils off first and the ether linked dimer fatty acid distills off later.

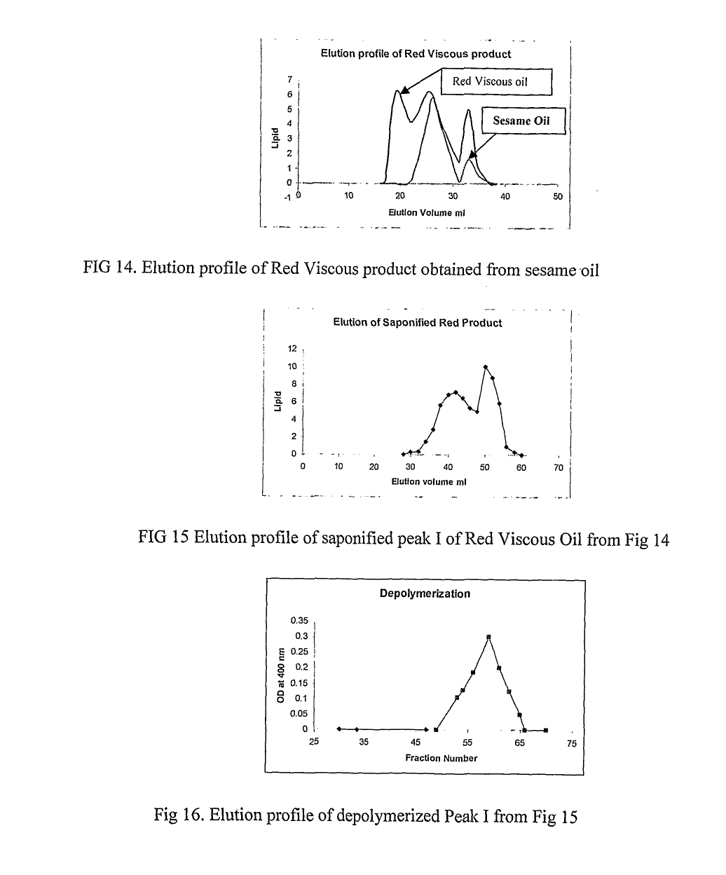

Gel permeation chromatography of the red viscous liquid is shown in FIG. 14. The red viscous liquid gave three fractions on Sephadex LH20. Fraction II and Fraction III were similar to those found in the elution of the starting material namely sesame oil without any treatment. Fraction II was found to be triacyl glycerol from TLC and fraction III was unesterified fatty acid.

Fraction I was subjected to saponification with 0.5 N alcoholic KOH followed by acidification with 0.5 N HCl. The lipids were extracted into diethyl ether. The ether was evaporated and the saponification products were dissolved in ethanol and subjected to gel filtration on Sephadex LH 20.The elution profile is shown in FIG. 15.

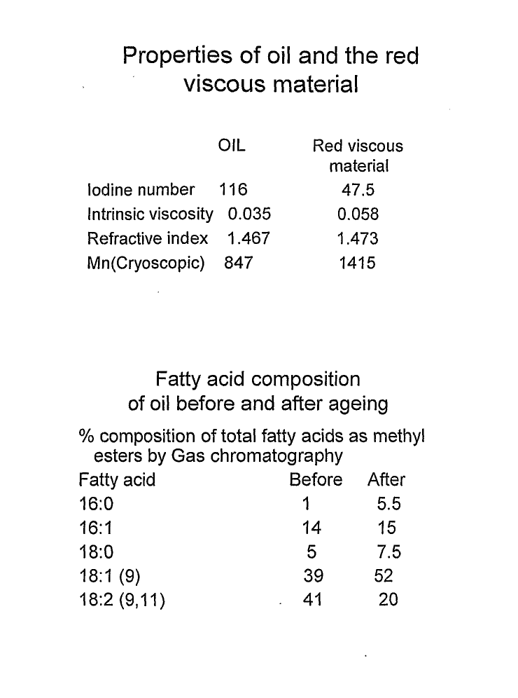

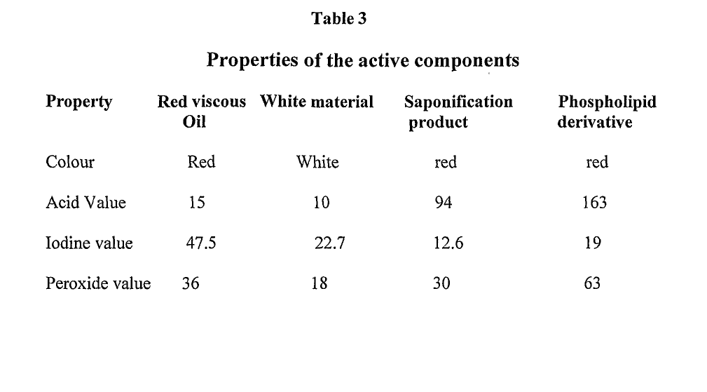

Two lipid fractions were eluted from the column. The second fraction corresponded to fatty acid by TLC. The first fraction was reacted with hydriodic acid in glacial acetic acid. The reaction products were then reduced by adding zinc dust to the reaction mixture. The elution profile of the lipids on Sephadex LH20 is shown in FIG. 16.

Peak I of FIG. 15 was reacted with Hydriodic acid and after reduction with Zn-acetic acid was eluted from sephadex LH 20 column. (First point at 30 ml represents Void volume of column, second point represents the elution of Peak I in FIG. 14 and the third point represents the elution of Peak I of FIG. 15)

The lipids from the elution peak were methylated with diazomethane and subjected to gas chromatography. Stearic acid was obtained as the only fatty acid.

In order to confirm this, 12-Hydroxy stearic acid and ricinolic acid (12 hydroxy oleic acid) were coupled as follows 0.2 mole of the hydroxyl fatty acid was methylated with diazomethane. This methyl ester was divided into two equal portions. To one portion (0.1 mol) in dry benzene a piece of sodium metal was added (100 mg) and the methyl ester of hydroxyl fatty acid was made to react at room temperature overnight.

To another portion of the methyl ester equimolar thionyl chloride was added and allowed to react at 60° C. for 30 min. It was cooled and the methyl ester treated with sodium metal was mixed. The mixture was heated at 60° C. for 1 hr and cooled. The lipids were extracted into diethyl ether and subjected to separation on TLC. The product of reaction remained at the origin while the unreacted fatty acid methyl esters moved towards the solvent front in a solvent system Hexane:Diethyl ether:Acetic acid 70:30:1 (by vol).

This product on reaction with hydriodic acid in glacial acetic acid followed by reduction gave stearic acid in both the cases were 12 hydroxy stedric acid was used or 12 hydroxy oleic acid (Ricinoluc acid) was used.

Taken together these results suggest that the fatty acids are linked through either linkage. Presence of the ether linkage was confirmed by nmr.

When the white product was saponified using 0.5N alcoholic KOH, followed by acidification and extraction as described for the red viscous product, a lipid fraction was obtained which on sephadex LH 20 gave two lipid fraction as shown in FIG. 15. The first fraction corresponded to the ether linked fatty acid while the second fraction corresponded to the fatty acid. The ether linked fatty acid could be depolymerized to stearic acid upon reaction with hydriodic acid followed by reduction

### 2. Demonstrating the Efficacy of the Prepared Agents

### 2.1 Differential Toxicity

In order to demonstrate the differential cytotoxic effects of the prepared agents, invitro experiments were performed using cultured cells. Both primary culture and cell lines were used in the study.

To demonstrate differential cytotocity, normal cells like HDCS, and VERO and transformed cells like HeLa, KB and MSF-8 were used.

Since transformed cells divide rapidly and do not have contact inhibition, a model of normal cells dividing rapidly was used in the study. This is a primary cell culture prepared from chick embryo.

In order to test the functionality of the prepared agents (the red oil obtained from the air oxidized oil and the ether linked fatty acid dimmer obtained after saponification of the red liquid) to distinguish between normal and transformed cells, a mixed culture was prepared consisting of HeLa and HDCS. When the cells were confluent, they were treated with the prepared agent. The left over cells were tested for their ability to be agglutinated by phytohemoagglutinin.

The cytotoxicity of the prepared agents on cancer cells in vivo was tested using a mouse model. Yoshida sarcoma was grown in the peritoneal cavity of swiss mice. Third day after the inoculation of the tumour cells, the prepared agent was injected into the peritomal cavity of the mice in one group and intradermal in another group. The differential action on the growth of tumour was measured in comparison with untreated tumour control. Normal cells in culture were transformed using viruses and ability of the prepared agent to kill the transformed cells was tested.

For comparable effects using the red oil, it was necessary to use about 10 times the quantity of the ether linked fatty acid dimer.

2.1.1 Differential Cytotoxic Effect with Potential for Cancer Treatment:

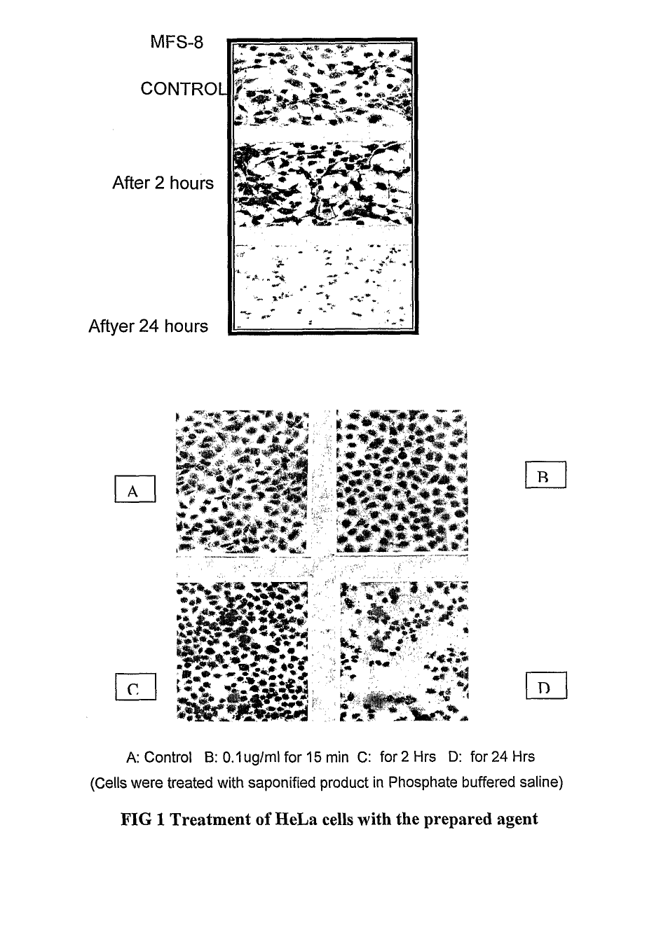

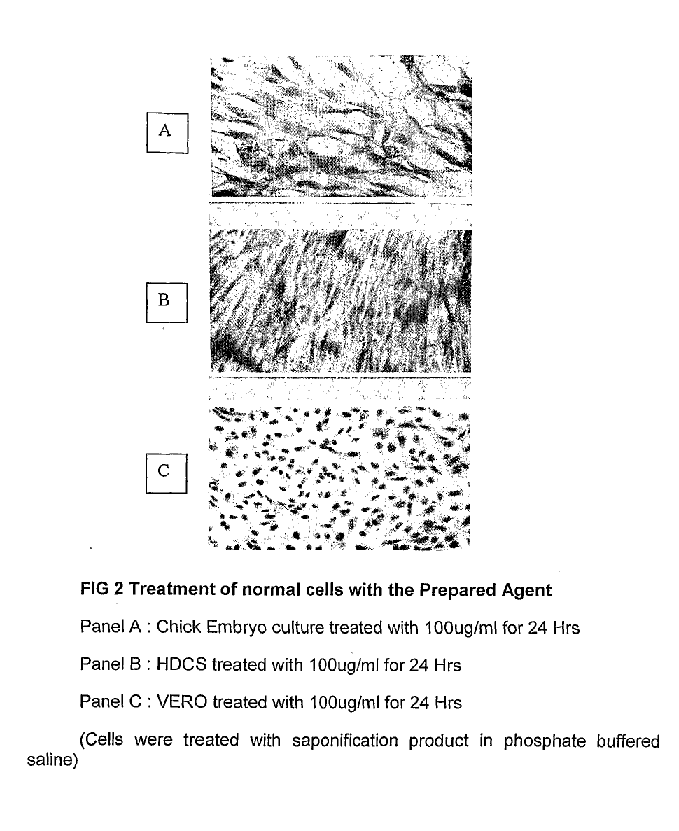

The ether linked fatty acid dimer or the red oil was cytototxic to transformed cells like HeLa, KB and MFS-8 at 0.1 μg/ml whereas it did not show any cytotoxic effect on HDCS and VERO at 100 μg/ml concentration (FIG. 1 -2). When the red oil was used, similar effects were observed except that the amounts of the red oil used had to be higher than when the ether linked fatty acid dimer was used. The red oil or the ether linked fatty acid dimer was also not cytotoxic to chick embryo culture at 100 μg/ml (FIG. 2).

There was no difference in the action of red oil or the prepared agent on cytotoxicity either to transformed cells or normal cells when tested under identical conditions. However the maximum cytotoxic effect with the ether linked fatty acid dimer was 0.1 μg/ml whereas the air oxidised oil, i.e. the red liquid was 10 μg/ml. The original starting oil was not cytotoxic.

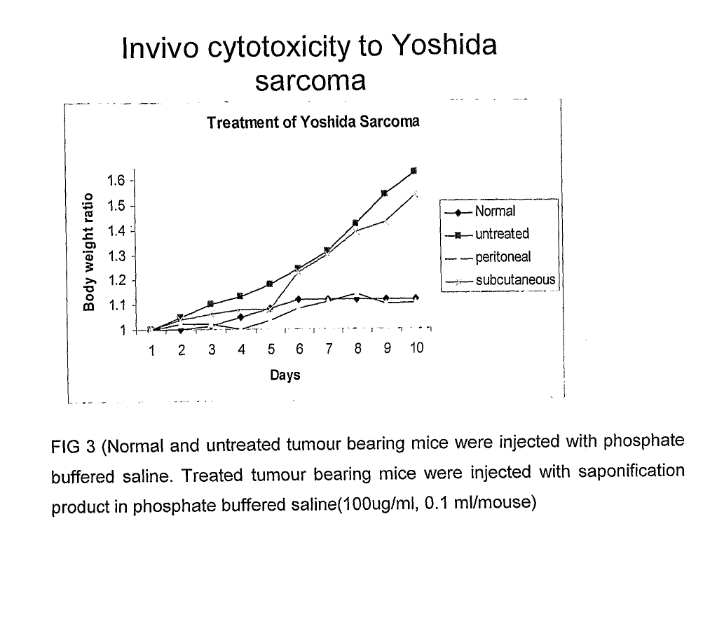

The effect of prepared agent on Yoshida sarcoma in mice is shown in FIG. 3.

When the tumor was treated with either the ether linked fatty acid dimer or red oil the effect was similar only when injected into the site of the tumor. However when injected subcutaneously it showed no effect.

For comparable effects using the red oil, it was necessary to use about 10 times the quantity of the ether linked fatty acid dimer.

### 2.2 Effect of the Prepared Agent on Skin Diseases:

The prepared agent and a placebo controlled double blind study were carried out.

The subjects were chosen from among the chronic dermatitis patients who had the disease for 5 years and longer. The subjects were divided into two groups of which one group received the placebo made from the untreated sesame oil in a carrier Lanoline and the second group received the red oil in the carrier Lanoline. Chronic dermatitis patients were asked to apply the ointment three times a day.

In order to unravel the mechanism of action on skin, in vitro and in vivo treatment were done on rat skin.

Rats were depilated on the abdomen one set of rats was used as control and the other set was treated with the red oil for 10 days once daily. The rats were subjected to gross physical examination for sign of irritation, scratch marks, inflammation or any other reaction. After sacrificing the rats, the blood was examined for specific signs of toxicity. Skin from control and treated portion of the skin was fixed in Bouins fixative, sectioned and stained to measure the thickness of the epidermis.

Newborn mouse ear was cut and placed in organ culture. It was painted on one side with either the red oil or the ether linked fatty acid dimer. The culture medium was changed on every third day. The skin was then fixed in Bouins fixative and sectioned. The thickness of the epidermis was measured.

The skin constituents of treated and control skin were determined. The skin was also used to measure conversion of ³H progesterone to hydrocortisone.

Incorporation of the ¹⁴C acetate into lipids of control and treated skin was determined.

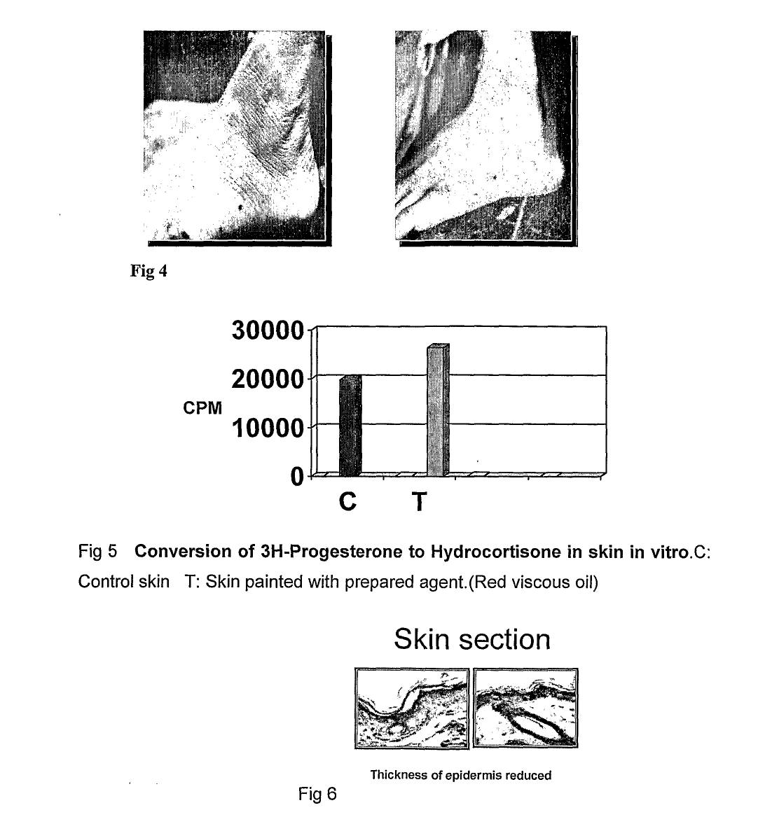

In the placebo controlled double blind study 23 patients reported relief from itching within 3 days. 15 patients reported that itching subsided within one week. After one month the skin was normal in these cases (total of 38 patients) 4 patients had relief from itching within one week and skin was normal after one month but had occasional relapse. 4 patients had symptomatic relief as long as the ointment was applied. Even after two months the skin had not returned to normal and 2 patients had no relief. All the patients were available for follow up. 27 patients had relief. The patients whose skin returned to normal were not available for follow up. 25 patients who had received placebo did not find any relief. They discontinued the use of the ointment after one week. Out of the 100 patients in this study 75 received the active ointment. Out of these only 6 patients did not show any cure. A typical case is shown in FIG. 4

The gross examination of rat skin treated with the red oil did not show any scratch marks or any other marks of inflammation or toxicity. The blood picture also did not show any signs of toxicity. The mucopolysacchardies and glycoproteins in the skin on treatment decreased, Δ⁴ 3-oxo steroids in the skin increased. Conversion of ³H progesterone to hydrocortisone also increased. Incorporation of ¹⁴C acetate into phospholipids of treated skin increased compared with the control (FIG. 5) the thickness of the control skin was 30.7±2.2 μm whereas that of treated skin was 17.5±1.4 μm (FIG. 6).

For comparable effects using the red oil, it was necessary to use about 10 times the quantity of the ether linked fatty acid dimer.

### 2.3 Testing the Effectiveness on Ageing

Rat skin was treated in vitro and in vivo with white material as well as the ether linked fatty acid dimer.

The skin of rats was depilated, and treated with the aged oil once daily for 10 days. The skin was then removed and one protion was fixed in Bouin's fixative. The other portion was tested for muropoly saceharides, glycoprotein and hormonal steroids.

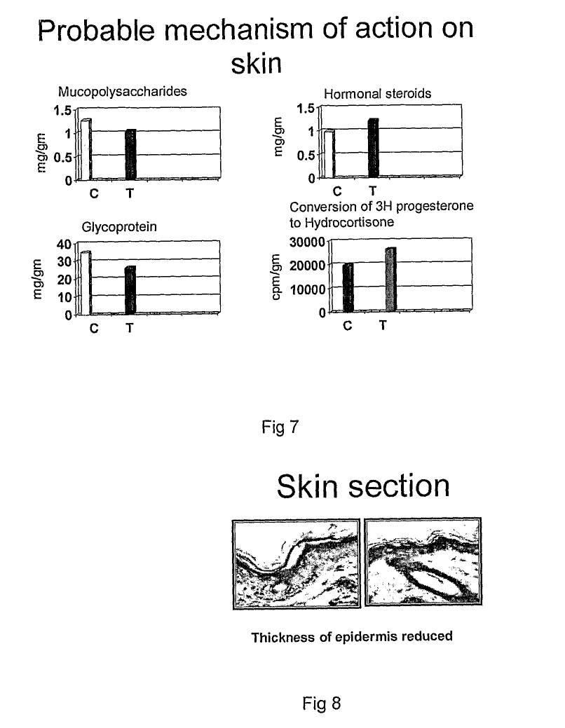

The mucopolysaceharides, glycoprotein and Δ⁴, 3-oxo steroids in the control and treated skin is shown in FIG. 7. The mucopolysacchardies and glycoproteins in treated skin had decreased and the Δ⁴, 3-oxo steroids increased.

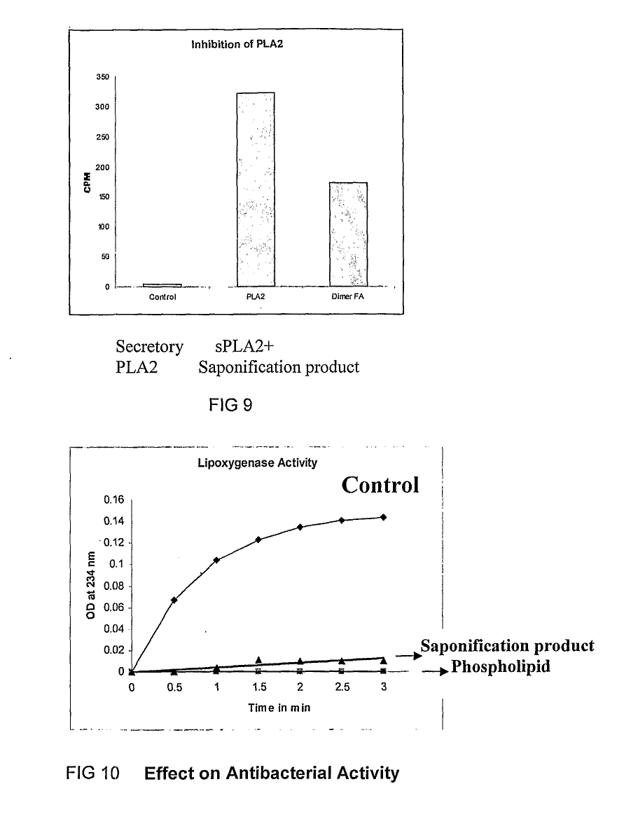

The thickness of the control epidermis was 30.7±2.2 μm and that of treated epidermis was 17.5±1.4 μm. Similar results were obtained from skin treated in vitro in organ culture (FIG. 8).

For comparable effects using the red oil, it was necessary to use about 10 times the quantity of the ether linked fatty acid dimer.

### 2.4 Effect of the Prepared Agent on Inflammatory Reactions

Purified synovial fluid (inflammatory) PLA₂ was used as the target enzyme. The substrate was E coli membrane having radio labeled fatty acid. The enzyme activity in the presence and absence of ether linked fatty acid dimer or the red oil was measured.

The ether linked fatty acid dimer or the oil was also used as an inhibitor in the lipoxygenase assay.

The ether linked fatty acid dimer inhibited PLA₂ by 45%. It also inhibited lipoxygenase by over 90% (FIG. 9,10). For comparable effects using the red oil, it was necessary to use about 10 times the quantity of the ether linked fatty acid dimer.

Bacterial culture was prepared and grown on a Petridish. A central well was made and 10 μl of ether linked fatty acid dimer was added. Norfloxacine disc was added on the dish for comparison. The ether linked fatty acid dimer was mixed with Vaseline and applied on acne for two individuals and one individual with atopic eczema.

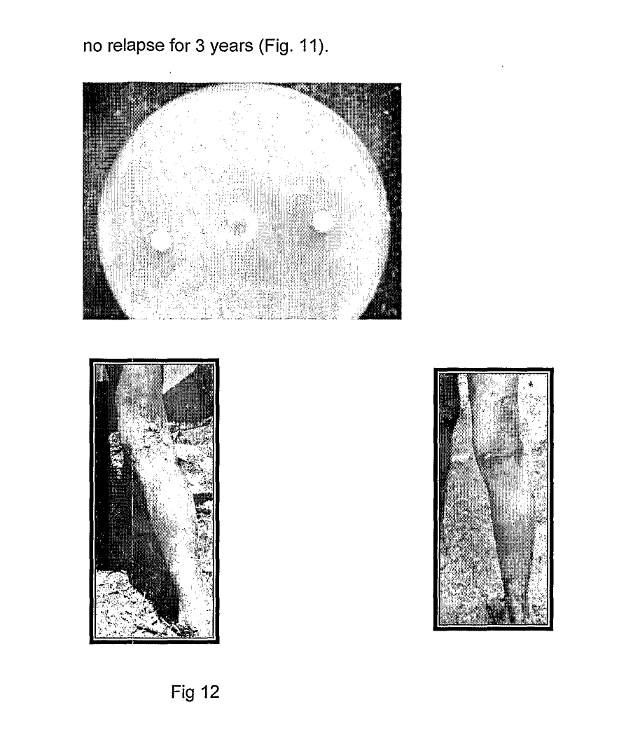

The zone of inhibition by prepared agent was larger that of norfloxacine (FIG. 11). It also cured the acne of two individuals. The atopic eczema was cured and had no relapse for 3 years (FIG. 12).

For comparable effects using the red oil, it was necessary to use about 10 times the quantity of the ether linked fatty acid dimer.

### 2.6 Effect of the Prepared Agent on Antifungal Activity

Fungal culture was grown on agar plate and the ether linked fatty acid dimer or the red oil was added on the plate and zone of inhibition was measured.

The ether linked fatty acid dimer or the red liquid was mixed with Vaseline and treated on athletes' foot and other fungal infections.

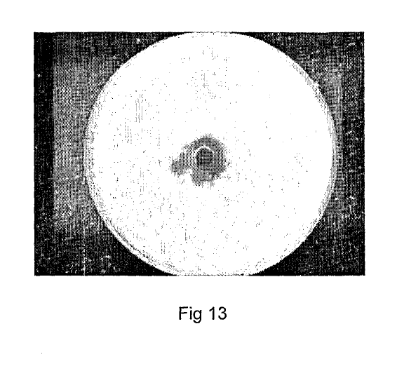

The prepared agents inhibited the growth of aspergellus (FIG. 13). It also cured 8 cases of fungal infection including 5 athletes foot infection.

For comparable effects using the red oil, it was necessary to use about 10 times the quantity of the ether linked fatty acid dimer.

1. Agents for the treatment of ailments and dysfunctions wherein
the agents are prepared by air oxidation of natural oils optionally in the presence of a catalyst wherein the isolated agents have Iodine value 40-60% of the starting oil, Saponification value 20-60 higher than that of the starting oil, Peroxide value 2-3 times that of the starting oil.

2. Agents of claim 1 which on saponification and acidification yields free fatty acids and ether linked fatty acid dimer.

3. A process for the preparation of the agents including the ether linked fatty acid dimer of claim 2, comprising steps of
ageing the oil in a vessel optionally layered with carbon particles,
allowing air oxidation with or without bubbling of air/oxygen optionally in the presence of catalysts to produce a red liquid of Iodine value 40-60% that of the starting oil, Saponification value 20-60 higher than that of the starting oil, Peroxide value 2-3 times that of the starting oil, and
separating the red liquid, optionally saponifying the red liquid, acidifying and isolating the ether linked fatty acid dimer.

4. The process for the preparation of agents including the ether linked fatty acid dimer of claim 3 wherein the red liquid in the oil is allowed to undergo air oxidation to produce a white precipitate having Iodine value of 40-60% that of the starting oil, saponification value 20-50 higher than that of the starting oil and Peroxide value of 40-60% of the red liquid.

5. The process of claim 3, wherein the oil is selected from the group consisting essentially of sunflower oil, safflower oil, ground nut oil, soybean oil, sesame oil and other unsaturated oils, preferably sesame oil.

6. The process of claim 3, wherein the oxidative process is catalysed by activated clays, metal oxides such as ferric oxide, lead oxide, mercury oxide, wherein lead oxide is the preferred choice, calcium octoate and potassium octoate.

7. The process of claims 3, wherein sesame oil with Iodine value of 60-70, saponification value of 220-230 and peroxide value of 10 and intrinsic viscosity of 0.035 on air oxidation produces a red liquid with iodine value of 30-40, saponification value of 250-270, peroxide value of 30-50 and Intrinsic viscosity of 0.05-0.07.

8. Agents including the ether linked fatty acid dimer of claim 2 wherein the agents at levels >0.1 μg/ml exhibit cyto-toxicity on transformed cells like HeLa, KB and MFS-8 and up_to 100 μg/ml do not exhibit cyto-toxicity on HDCS and VERO.

9. Agents of claim 1 wherein the agents at levels of 0.1 μg/ml-100 μg/ml significantly control conditions of Yoshida sarcoma.

10. Agents of claim 1 to reduce the levels of mucopolysacchardies and glycoproteins specially in skin cells, increase levels of Δ⁴ 3-oxo steroids and aid in the conversion of ³H progesterone to hydrocortisone.

11. Agents of claim 1 inhibit enzyme activities such as PLA₂ and lipoxygenase activity, growth of fungus such as aspergellus.

12. Compositions comprising the agents of claim 1 for conditioning of skin by reducing the levels of mucopolysacchardies and glycoproteins, increasing the levels of Δ⁴ 3-oxo steroids and aiding in the conversion of ³H progesterone to hydrocortisone.

13. A medicament comprising the agents of claim 1 for treating diseased conditions in mammals such as uncontrolled cell growth, tumors, skin aliments, fungal infection, athlete's foot infection, acne and atopic eczema.

14. A method of treating sarcoma using the medicament of claim 13 comprising administering a medicament, wherein the agents at levels >0.1 μg/ml exhibit cyto-toxicity on transformed cells like HeLa₁ KB and MFS-8 and up to 100 μg/ml do not exhibit cyto-toxicity on HDCS and VERO.
